(19) United States
(12) Patent Application Publication
(10) Pub. No.: US 2021/0023393 A1
(43) Pub. Date: Jan. 28, 2021

(54) Stacked assembly of 2D radiochromic dosimeters to provide 3D dosimetric data

(71) Applicant: John A. Adamovics, Skillman, NJ (US)

(72) Inventor: John A. Adamovics, Skillman, NJ (US)

(21) Appl. No.: 16/523,186

(22) Filed: Jul. 26, 2019

(51) Int. Cl. A61N 5/10; A61B 90/00; G01T 1/06

(52) U.S. Cl. CPC A61N 5/103; A61B 90/39; A61B 2090/3937; G01T 1/06; A61N 2005/1076; A61N 5/1071

(57) ABSTRACT

The invention relates to a polymeric dosimetric sheet with a thickness of 0.8 to 10.0 mm which contains a radiochromic dye and other additives and which reacts with x-ray or other ionizing radiation to form a stable color. The invention also relates to a stack of the dosimetric sheets which constitute a three-dimensional dosimeter. Upon irradiation, the sheet stack captures the radiation field of an applied radiation treatment plan. The irradiated sheet stack is disassembled and scanned with readily available devices to afford an array of two-dimensional images which can verify the treatment plan throughout the entire target volume by computerized methods.

### REFERENCE TO RELATED APPLICATION

This non-provisional utility patent application claims benefit of provisional patent application 62/711,561 filed Jul. 29, 2018 under 35 U.S.C. § 119(e).

### FIELD OF THE INVENTION

The invention relates to a three-dimensional (3D) dosimeter comprised of an array of two dimensional (2D) dosimetric sheets with precisely controlled dimensions and high-quality optical surfaces which are assembled and confined in a stacked array. The assembled stack provides a 3D dosimeter with a very high percentage of high-resolution dosimetric material throughout its volume. The invention is useful in measuring a 3D field of radiation in a wide variety of applications. In one non-limiting application, the invention is useful in the treatment planning of radiation therapy for the treatment of cancer.

### BACKGROUND OF THE INVENTION

More than 14 million new cases of cancer are diagnosed globally each year. Radiation therapy (RT) has the potential to improve the rates of cure of 3.5 million people and provide palliative relief for an additional 3.5 million people. These estimates are based on the fact that approximately 50 percent of all cancer patients can benefit from RT in the management of their disease. Approximately half of these may receive a level of curative care by RT (Jaffray, D. A. and Gospodarowicz, M. K., “Radiation Therapy for Cancer,” in Cancer: Disease Control Priorities, Third Edition (Volume 3); Gelband H, Jha P, Sankaranarayanan R, et al., editors. Washington: The International Bank for Reconstruction and Development/The World Bank; 2015, Chapter 14). Because radiation affects normal tissue as well as tumor tissue, it is imperative to achieve a high level of control of the volume of incidence and strength of dose of radiation during RT procedures. The goal of radiation oncologists and medical physicists in the treatment planning and delivery of RT is to irradiate the localized tumor mass to the highest possible extent while minimizing the exposure of nearby healthy tissue, particularly when the tumor is proximal to tissues with limited radiation tolerance, known as critical normal structures.

Currently, complex radiation treatments such as Intensity-modulated radiation therapy (IMRT, reviewed by Hong, T. S. et al British Journal of Cancer, (92)1819-1824, 23 May 2005), Volumetric modulated arc therapy (VMAT, reviewed by Teoh, M. et al, British Journal of Radiology; 84(1007): 967-996, Nov. 2011), and Stereotactic ablative radiation therapy (SBAR, Chang, J. W., Annals of Translational Medicine 31(1): 12, Jan. 2015) provide improved dose distribution and conformity over the target volume by delivering a complex array of radiation with several dose intensities and angles of incidence.

These specialized complex RT modalities, while superior in delivering radiation doses in a gradient of intensities to tumor tissue at various angles within the target volume, present a difficult challenge for verification of the planned radiation treatment. Treatment plan verification is an essential step in the RT process, and is necessary to map the planned treatment dose to the actual delivered dose throughout the treatment volume, so that the radiation oncologist can be confident that the planned RT protocol will maximize tumor exposure while limiting toxicity to normal tissues when delivered to the patient.

An optimal treatment verification system would include a dosimetric device capable of measuring dose depth and intensity throughout the entire target volume. Such a dosimetric device should provide, throughout the target volume, linear response to the absorbed dose over a wide dose range, tissue equivalency over a wide energy range, and the capability to provide 3D dose distribution with high resolution.

The invention disclosed herein is an improvement over dosimetric devices currently utilized in RT planning protocols. The invention relates to a stacked array of 2D sheet dosimetric media of controlled uniform thickness precisely aligned over a determined height to provide an essentially solid 3D dosimeter of clinically relevant volume. After irradiation, the dosimeter is disassembled and each individual 2D sheet is scanned in precise orientation with a cost-effective widely available flatbed scanner to provide an array of high-resolution dosimetric data, which is then compared with the treatment plan. The device of the invention provides linear response to the absorbed dose over a wide dose range, which is not dependent on the radiation energy, nor on radiation dose rate and has the capability to provide 3D dose distribution with high resolution (Wang, Y. et al, An Investigation of Dosimetric Accuracy of A Novel PRESAGE Radiochromic Sheet and Its Clinical Applications, 10th IC3DDose Conference, Duke Kunshan University, China, Sep. 16-19, 2018).

### DESCRIPTION OF THE RELATED ART

Several dosimetric methods have been developed to map dose delivery in the verification of radiation treatment plans. Simplest of these are methods employing a single planar two-dimensional (2D) dosimeter which, when placed in the planned treatment field, provides a map of radiation incident at one particular plane within the treatment volume. Dosimetric materials used in these 2D methods include radiographic films, radiochromicfilms, and radiochromic plastic films. In contrast, 3D dosimeters with the capability of measuring the impact of radiation on two or more regions of a planned target volume, or essentially the entire target volume, have been utilized. 3D dosimetric materials include stacked films, 3D gel dosimeters, 3D solid plastic dosimeters, and arrays of electronic radiation sensors. Radiation sensors, such as thermoluminescent dosimeters, scintillation detectors, and semiconductor detectors, are not germane to the present invention and are not herein considered relevant related art.

Radiographic films which are 2D dosimeters are made of emulsions typically employing silver halides and other components in gelatin adhered to a polymeric base, typically cellulose or polyester. These films are useful in the measurement of radiation incident to single plane selected by the dosimetrist within the target volume. Radiographic films require chemical development, much like photographic films, and, once developed, are a permanent record of the irradiation event. These films have a dosimetric emulsion layer of 10-20 μm, and their use represents a sampling of one plane within the target volume. An overview of radiographic films has been published (https://www.aapm.org/meetings/09SS/documents/26DAS-RadiographicFilm.pdf, accessed Jul. 17, 2018).

Related 2D radiochromic films when irradiated result in color development, and no chemical treatment is required prior to image evaluation by a spectrophotometer or densitometer. Radiation-sensitive molecules which might be used in radiochromic plastic films include diacetylenes, which polymerize upon irradiation to provide optical changes, pH sensitive dyes, and radiochromic dyes. Thicknesses of the dosimetric layers range from 6.5-38 μm, and their use represents a sampling of one thin plane within the target volume An overview of radiochromic films has been published (http://amos3.aapm.org/abstracts/pdf/99-27719-359478-112521-1197135036.pdf, accessed Jul. 17, 2018).

Radiochromic films arrayed in parallel have been used in to evaluate radiation fields at several planes within the target volume. McCaw et al (Med. Phys. 2014 May; 41(5):052104. doi: 10.1118/1.4871781) reported the use of a stack of 22 GafChromic EBT2 films (Ashland Specialty Ingredients, Wilmington, Del.) separated by 1 mm Virtual Water (Med-Cal, Verona, Wis.) spacers held within a Virtual Water cylindrical phantom. The dosimeter size was 38.3 mm (diameter)×27.2 mm (height). The active layer in EBT2 film is 28 μm, or 0.028 mm (Niroomand-Rad, A., 57th Annual AAPM Meeting Abstracts, 2015, amos3.aapm.org/abstracts/pdf/99-27719-359478-112521-1197135036.pdf; accessed Jul. 28, 2018). Over the height of the dosimeter, the 22 EBT2 films contributed 22×0.028 mm=0.616 mm, while the remainder of the height, composed of spacers, adhesive layers, and polyester film support contributed 27.1 mm. While the dosimetric films were evenly dispersed throughout the volume of the dosimeter, the portion of the target volume sampled was 0.616/27.2 or 2.3%. Thus, stacked radiochromic films provide a dosimetric report of only a small fraction of the target volume. In addition, EBT films have limitations and disadvantages which adversely affect accuracy and precision, including non-linear dose response, non-uniformity, and heat sensitivity (Lynch, B. D. et al, Medical Physics, 16 Nov. 2006, https://doi.org/10.1118/1.237050, accessed Jul. 24, 2019). The dosimetric sheets of the invention overcome these limitations.

In U.S. Pat. No. 6,826,313 to Robar et al, a dosimetric system was disclosed in which a plurality of “spaced apart” two-dimensional analog sensors formed a plurality of two-dimensional analog images to be scanned to form digitized images (Column 10, line 63). The invention clearly relates to films or other “analog sensors” intended a sample only a portion of the irradiated volume by providing “a series of two-dimensional images representing the integrated dose provided in different planes through the dose distribution being studied.” (Column 5, line 51). The inventors concede “not all cells of the data structure will necessarily correspond to points which are imaged on one of the films 12. Many parts of the 3D distribution are in between adjacent films 12.” (Column 7, line 23). Although the '313 patent is silent as to the magnitude of space separating analog sensors, the inventors acknowledge that many portions of the volume are not measured and they rely on interpolation algorithms to populate the distribution volume with data calculated from a bicubic interpolation (Column 7, line 35).

3D chemical dosimeters are designed to measure the entire target volume. Various 3D dosimeters which are water based have been described, including the Fricke, Fricke-xylenol orange, polyacrylamide polymer and related polymer gel, and micelle gel formulations. While these 3D dosimeters are designed to provide continuous integrated dose measurement through the target volume, post-irradiation measurement can be problematic. Gel dosimeters suffer from sensitivity to oxygen, reproducibility issues and image stability. The dosimetric gel, which is fluid, must be prepared, stored, and used within a container, which may present optical aberration issues. Gel dosimeters must be analyzed by magnetic resonance imaging, x-ray computed tomography, or optical tomography. Instruments for such analyses are costly and are often only available in regional cancer centers. A second type of 3D dosimeter does not contain water but is based on polyurethane which eliminate many of the problems of 3D gel dosimeters have developed. Solid polyurethane dosimeters produce, upon irradiation, an accurate and reproducible image across essentially the entire volume of the transparent dosimeter (U.S. Pat. No. 9,357,925 to Adamovics). These plastic solid dosimeters capture the applied radiation field but must be analyzed by optical computed tomography. Optical CT instrumentation is costly and is often only available in regional cancer centers. 3D chemical dosimeters have been reviewed (Schreiner, L. J., Journal of Physics Conference Series, 573: 012003, 2015 and references cited therein).

Radiochromic plastic articles have been reported but until the present invention workers have been unable to produce radiochromic plastic articles as sheets with defined, accurate, and uniform thickness, with high-quality optical surfaces.

Canadian Patent 2,495,304 to Patel discloses “a process of making radiation sensitive molded or casted shaped polymeric devices including coating, film, fiber, rod, plaque and block for monitoring radiation dose of UV, X-ray, gamma ray, electron, protons, alpha particles or neutron radiation prepared by solidification of molten polymer containing at least one radiation sensitive material capable of developing or undergoing a color, fluorescence, or opacity change, with or without activator, when contacted with UV, X-ray, gamma ray, electron, protons, alpha particles or neutron, and optionally additives such as UV absorber, convertor, surfactant and solvent” (p. 12, line 3). Although the '304 patent is primarily directed to the use of diacetylene indicators, the potential use of leuco dyes is disclosed (p. 21, line 20). The '304 patent does not teach a formulation or method to provide radiochromic coatings or films of defined uniform thickness or with high-quality optical surfaces. Example 2 (p. 37, line 19) of '304 patent discloses the use of leucomalachite green (LMG), with other additives, in a polymethylmethacrylate polymer matrix. In this example, the '304 patent is silent with respect to the dimensions or optical quality of the solid product, and states merely that “the irradiated portion turned green” (line 31), a phenomenon well-known to those with skill in the art. The inventor of the present invention has found that formulations containing leuco dyes appropriate for fabricating sheet dosimeters must be kept at temperatures less than 25° C. during mixing and molding steps to avoid unwanted premature thermally-induced coloration. The '304 patent teaches away from this finding in Example 4 (p. 33, line 19) in which LMG is subjected to 120° C. for an undisclosed period of time.

Dumas, M. and Rakowski, J. T. published an article examining “Presage dosimeter formulations in sheet form” (Medical Physics 42, 7138 (2015); doi: 10.1118/1.4936104) in which LMG, bromoform, and optionally a tin catalyst were formulated in polyurethane and cast into an aluminum mold. The authors recognized the potential of polyurethane sheets containing a leuco dye reporter molecule as dosimetric tools but admitted the failure of their attempts to produce sheets of adequate quality. They conceded that their process of forming the sheet, the formulation being “cast into an aluminum mold” was unable to produce high-quality sheets. No description of the aluminum mold, nor teaching as to the details of the casting process are offered. They concluded that the “simple equipment used in the fabrication process . . . limited the dosimeter's sensitivity uniformity by agglomeration of air bubbles in the material, nonuniform concentration of chemicals throughout the material, and thickness variations”. The authors offered no instruction or guidance on how to make improvements other that the need for “improvements in mixing tools and molds”. The authors also suggest that higher sensitivity of the dosimetric sheets would be advantageous in Quality Assurance of complicated radiotherapy plans, but give no insight or guidance as to how this might be achieved. The authors also speculate that “Sheets could be stacked together and setup parallel to the beam. This would give a much higher spatial resolution in the beam direction than Presage's current 3D use in proton dosimetry”. The authors provide no guidance or suggestions as to methods to control sheet thickness and quality, formulation sensitivity, or ways in which individual sheets might be accurately “stacked together” to provide a useful 3D dosimeter.

Polyurethane matrices containing LMG were prepared in a rectangular microtiter plate Youkahana, E. Q. et al (Biomed. Phys. Eng. Express 2 (2016) 045009; http://iopscience.iop.org/article/10.1088/2057-1976/2/4/045009; accessed Jul. 24, 2018). The authors simply poured a polyurethane formulation into microplate wells and assumed gravity would cause levelling to a uniform thickness before the formulation cured to a solid. No attempt to remove the polyurethane sheets from the polystyrene plates was reported, and the authors gave no suggestion that dosimetric polyurethane sheets freed from the polystyrene container would be useful. The authors acknowledge that their casting technique does not provide a sheet dosimeter of defined and uniformed thickness: “Although the [polyurethane] dosimeter was set in a plate designed specifically for the CLARIOstar microplate reader (BMG Labtech Cary, N.C.) the thickness of the dosimeter may not have a uniform thickness.”

An initial investigation of polyurethane sheet dosimeters (Jordan, K. and Adamovics, J., Med. Phys. 43:6 (June 2016), SU-F-T-550) concluded that the dosimetric sheets exhibited high sensitivity and linear dose response but suffered from up to 20% variability in thickness as measured across the sheets. The authors suggested that “improvements in mold design” might result in sheets with better dosimetric performance but offered no guidance or instruction how to achieve those improvements. The present invention provides the solution to this problem.

### SUMMARY OF THE DISCLOSURE

The invention provides a solid polyurethane tissue equivalent 3D dosimeter which captures applied doses of radiation with high resolution throughout the entire target volume of a radiation treatment plan. Radiation causes the change of a radiochromic leuco dye, distributed uniformly throughout the dosimeter, from a colorless form to a colored form in regions selected by a radiation therapy treatment plan to receive radiation within the target volume. The dosimeter of the invention is comprised of an assembly of sheets of polyurethane manufactured with precise uniform thickness, high-quality optical surfaces, precise length and width. The invention provides means to assemble the sheets into a stacked array of precise dimensions. After irradiation the dosimeter is disassembled, and each component sheet is individually scanned by a widely available flatbed scanner, providing a plurality of precisely aligned 2D images. The 2D images may be manipulated by a computer program to reconstruct a 3D image. The reconstructed image may be compared by a computer program with the radiation treatment plan. In addition, the 2D images may be sequentially compared to the radiation treatment plan with computer techniques well known by those skilled in the art.

### BRIEF DESCRIPTION OF THE DRAWINGS

FIG. 1 depicts components of a Sheet Mold in an exploded view.

FIG. 2 portrays an assembled Sheet Mold.

FIG. 3 shows an individual Sheet with precise dimensions, mounting apertures, and fiducial marks.

FIG. 4 illustrates the components of a Sheet Stack in an exploded view.

FIG. 5 depicts an assembled Sheet Stack and its orientation along three-dimensional axes

FIG. 6 depicts the anthropomorphic phantom STEEV (CIRS, Norfolk, Va.)

FIG. 7 illustrates the process of fabrication and use of a Sheet Stack Dosimeter.

### DEFINITION OF TERMS USED HEREIN

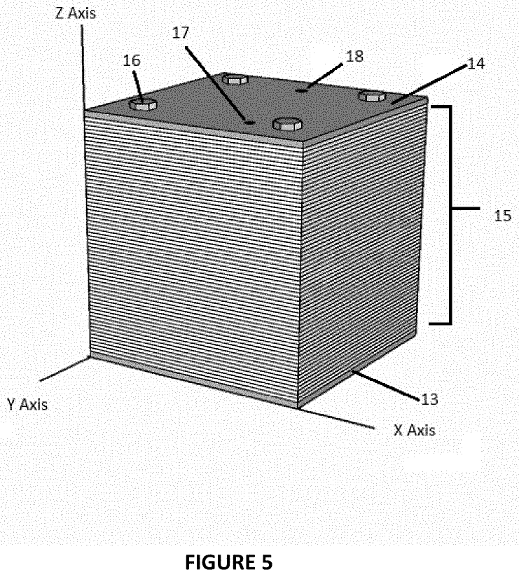

4,4′-(phenylmethylene)bis(N,N-dimethylaniline) also known as leuco malachite green (LMG)
4,4′,4″-methanetriyltris(N,N-dimethylaniline) also known as leuco crystal violet (LCV)
4,4′-((2,4-dimethylphenyl)methylene)bis(N,N-diethylaniline) also known as (2,4 Dimethyl-LMG-DMA)
4,4′-((4-isopropylphenyl)methylene)bis(N,N-diethylaniline) also known as (cumin-LMG-DEA)
4,4′-(o-tolylmethylene)bis(N,N-diethylaniline) also known as (2-Methyl-LMG-DEA)
4,4′-((4-methoxyphenyl)methylene)bis(N,N-dimethylaniline) also known as (4-MeO-LMG-DMA)
additive solvent: solvent used to impart desired attributes to the dosimetric sheet, for example to increase pliability or enhance color resolution and stability
clinical utility: regarding the usefulness of a device in measurement or evaluation of a radiation treatment plan or of delivery of radiation to a patient
color stability: the degree of permanence of a colored image formed within a dosimeter upon irradiation
cure: completion of the polymerization of a liquid formulation
demolding or release agents: a chemical to inhibit materials bonding to mold surfaces
endplate: polypropylene sheet used at the ends of the mold stack in a sheet dosimeter molding apparatus
flatbed scanner: an optical scanner which makes use of a flat surface for scanning
high-quality optical surfaces: surface of a film or sheet which is essentially free from blemishes, scratches, digs, protrusions, gaps, and fissures, or other blemishes
inner plate: polypropylene sheet used for form gaps in multichannel sheet molds. Inner plates are separated from each other and from mold endplates by spacers.
orientation axes: definition of 3D space, herein x, y, and z axes as illustrated in FIG. 5
percentage of formulation is defined as w/w.
plasticizer: additive to impart softness or pliability to a polymer
polymerization catalysts: for polyurethane, organomercury or organotin compounds which facilitate condensation of diisocyanate with polyol
polypropylene films: films with thickness in the range of 0.8 mm to 10 mm which are insensitive to radiation
post-curing modification: treatment by manipulation by cutting or machining sheet dosimeters to prepare for assembly into a stack after polymerization is complete
pot-life: length time which a liquid prepolymer formulation is workable before its viscosity increases to the point where it is no longer moldable
radiation treatment plan: the arrangement of the alignment, duration, and intensity of radiation to be delivered to a patient
target volume: volume of tissue containing known and suspected tumor mass
sheet dosimeters: radiochromic plastic sheets having thickness in the range of 0.8 mm to 10 mm
shim: polypropylene sheets of various thicknesses
spacer: “U” shaped inserts used in sheet dosimeter molding apparatus
tissue equivalent: Material designed to have interaction properties when irradiated similar to those of soft tissue

### DETAILED DESCRIPTION OF THE INVENTION

The invention disclosed herein is useful in the field of radiation therapy of mammalian diseases and relates to a 3D solid dosimeter which is capable of capturing a field of incident radiation throughout the volume targeted by a radiation therapy treatment plan. The solid 3D dosimeter is meant to assess with high resolution the dose level, depth, and distribution of therapeutic radiation within a target volume selected by the medical practitioner and defined by the needs of the patient. It is designed to evaluate and verify that a radiation treatment plan conforms to the design of the medical practitioner to focus treatment on diseased tissue while minimizing exposure of healthy tissue, prior to the actual radiation therapy event. The 3D solid dosimeter is comprised of a plurality of tissue-equivalent sheet dosimeters which are manufactured to precise dimensions of thickness, length, and width, and possess high-quality optical surfaces. Direct comparison to a calibration curve allows dosimetric sheets of the invention to provide absolute dose information across the entire volume of the dosimeter stack, a feature not found in other 3D dosimetric modalities. The sheet dosimeters contain, among other additives, a radiochromic leuco dye which transforms from colorless to a colored form upon exposure to radiation. Due to the adaptability of the invention, dosimetric sheets may be fabricated with lengths from 10 cm to 50 cm, and with widths from 10 cm to 50 cm. Sheets of clinical utility have lengths from 10 to 25 cm and widths of 1 to 25 cm. The sheet dosimeters can be produced in a variety of thicknesses. Sheet dosimeters may be produced in thicknesses ranging from 0.8 mm to 10.0 mm. Most useful sheet dosimeters will be fabricated to have thickness between 1.0 and 5.0 mm. A plurality of sheet dosimeters is machined, tooled, cut, modified, or transformed by processes well-known in the art so that each individual sheet dosimeter possesses means to align precisely with another and may be secured in a vertical array, one sheet dosimeter on top of another, to a height determined by the medical practitioner. The height of the stack of sheet dosimeters may be in the range of 1.0 cm to 20 cm. Sheet dosimeter stacks with the most clinical utility have a height between 2.0 cm and 15.0 cm. Sheet dosimeter stacks contain between 5 and 100 sheet dosimeters. The sheet stack dosimeter is placed in exact orientation to mimic the target volume of radiation intended for the patient, by means well-known in the art of radiation oncology. Radiation is applied per the radiation treatment plan, and the irradiated dosimeter is disassembled, providing a plurality of irradiated sheet dosimeters. The irradiated sheet dosimeters are individually scanned, for example by a readily available flatbed scanner, in a process which insures the exact order of the individual sheets along the axial (height) dimension is preserved. The resulting set of 2D dosimetric data is then manipulated to compare to the treatment plan.

Formulation. The chemical components of the sheet dosimeters of the invention are critical to the production of products with superior optical, mechanical, and chemical attributes. After extensive experimentation, it was found that by varying the nature of the prepolymers and the relative amount of leuco dye, initiator, solvent, and plasticizer in the formulation, the stability, resolution, dose response and hardness of sheet dosimeters could be adjusted. Thus, by proper selection of formulation components, the invention provides a plastic dosimeter matrix which can be molded into sheet dosimeters which overcome the limitations of radiochromic plastic films, or sheets of the prior art.

The sheets of the invention are made of polyurethane. The components of the formulation are thoroughly mixed with polyurethane prepolymer A (a diisocyanate) and prepolymer B (a polyol). It is well-known in the field of polyurethane manufacturing that the nature of the prepolymers and the nature and concentration of polymerization catalysts in commercially available polyurethane kits will determine the pot-life of a formulation. It is also well-known that once admixed, components of a polyurethane formulation with a long pot-life, which gives allows workers extended time for manipulations before curing begins, results in an extended curing time. It has been found that the optimal pot-life of the formulation for sheets is between ten and twenty minutes. This allows time for thorough mixing of components and introduction of the formulation into a mold, while providing acceptable curing times. Manufacturers of polyurethane casting kits (for example, BJB Enterprises, Tustin, Calif. and Smooth-On, Macungie, Pa.) offer products with various pot-life specifications.

The inventor has found that any of the class of triarylmethanes containing a dialkylamino substituent on two or three of the phenyl rings can be used as the leuco dye in the sheet formulation. Particularly useful are 4,4′-((phenyl)methylene)bis(N,N-dimethylaniline) and 4,4′-((phenyl)methylene)bis(N,N-diethylaniline) derivatives substituted on the phenyl ring not containing a dialkylamino group. A subset of these triarylmethane leuco dyes, herein defined as “sheet leuco dyes” are most useful in the invention. These sheet leuco dyes are substituted on the phenyl ring not containing a dialkylamino group with hydrogen, alkyl, alkoxy, halogen, or aryl groups.

As a non-limiting illustration, selection of 4,4′-((2,4-dimethylphenyl)methylene)bis(N,N-diethylaniline), 4,4′-((2,4,5-trimethylphenyl)methylene)bis(N,N-dimethylaniline), or 4,4′,4″-methanetriyltris(N,N-dimethylaniline) as the leuco dye component of the sheet formulation, when coupled with the choice of the appropriate initiator, solvent, and plasticizer, provides according to the method of the invention a sheet product which, upon irradiation, exhibits a stable, high-resolution colored image. The sheet leuco dye comprises between 0.5% to 10.0% of the formulation.

It has been reported that radical initiators facilitate the transformation of a leuco dye within a plastic matrix to a colored form upon irradiation (U.S. Pat. No. 7,098,463 to Adamovics). A radical initiator is a critical element of the formulation of the sheets of the invention. Although other radical initiators may be useful in the practice of the invention, it has been found that polyhaloalkanes, trihalomethanes, and tetrahalomethanes are cost-effect agents which give satisfactory results. Particularly useful are bromoform and carbon tetrabromide. The radical initiator comprises between 0.1% to 5.0% of the formulation.

The addition of organic solvents to the sheet formulation may serve two purposes. The leuco dye is sparingly soluble in both of the polyurethane prepolymers. In order to achieve a homogeneous distribution of the leuco dye throughout the entire volume of the sheet dosimeter, it is necessary to dissolve the dye in an appropriate, compatible solvent. A wide variety of organic solvents, including but not limited to, ketones, haloalkanes, arenes, sulfoxides, tertiary amines, and heterocyclic lactams, might be used to solubilize the leuco dye prior to thorough mixing with one of the prepolymers. Particularly useful are dichloromethane, dimethyl sulfoxide, tetrahydrofuran, and N-methyl-2-pyrrolidinone. Solvent to dissolve the leuco dye comprises between 3% and 15% of the formulation.

The use of a second solvent as an additive provides enhancement of the color image after irradiation of the sheet. Surprisingly, it was discovered that a small amount of a ketonic solvent added to the formulation resulted in sheets with higher dose sensitivity. Appropriate additive solvents include dialkyl ketones, aralkyl ketones, and carbocyclic ketones. Particularly useful are 5-nonanone, cyclohexanone, and methyl ethyl ketone. The additive solvent comprises between 0.5% and 10% of the formulation.

It was discovered that the addition of a plasticizer or softener to the sheet formulation conferred several advantages to the casting, demolding, and sheet stack assembly aspects of the invention. Formulations containing plasticizer have an increased pot-life, which facilitates the thorough combining of the formulation components and manipulation of the formulation prior to the molding step. Addition of a plasticizer decreases the viscosity of the formulation which aids in both pre-molding manipulation and in introduction of the formulation into the sheet mold.

It was discovered that addition of plasticizer improved the demolding process, as the cured sheet had less tendency to adhere to the mold sides. A further advantage is the high optical quality of the x-y planar surfaces of sheets prepared from formulations containing plasticizer. Choice of the concentration of plasticizer in the formulation allows the modulation of the rigidity of the sheet produced. Thus, by selection of an appropriate amount of plasticizer, a pliable sheet is produced. Individual pliable sheet dosimeters may have utility in in vivo dosimetry (Dumas and Rakowski, 2015 vide supra; Adamovics et al, unpublished results). Thus, sheet dosimeters might be used as bolus materials for use in radiotherapy. Sheet formulations provide tissue-equivalent polymeric materials which can modulate radiation and therefore act as a bolus to increase surface dose while allowing a homogeneous dose to a target volume, while at the same time provide quantitative measurement of the applied radiation. It was found that, in addition to the aforementioned advantages, sheets with a minimal element of pliability possess less hardness, and as a result post-curing modification is facilitated. Sheets with less hardness can be more easily cut to precise x-y planar dimensions, engraved with fiducial marks, and fitted with mounting apertures.

It will be appreciated that an assembly of sheets with a slight element of pliability, when pressed together between a rigid top endpiece and a rigid bottom endpiece through the action of four compression bolts, will be forced into a coplanar relationship, so that any slight planar inhomogeneities in the individual sheets will be effectively eliminated, thereby obviating possible optical artifacts which may occur in a similarly compressed stack of rigid sheets.

Mold design. The materials and method of assembly of the mold are critical elements in the fabrication of dosimeter sheets with specified, uniform thickness and with high-quality optical surfaces. Molding of polyurethane dosimeters in sheet form was reported (Dumas and Rakowski, vide supra) with disappointing results. The authors utilized an aluminum mold but are silent as to dimensions or means to control the thickness of the product. It has been found that polyurethane formulations molded in aluminum tend to adhere to the metal. As a result, demolding or release agents may be required which leave an unacceptable residue on the product which is difficult to remove. In the absence of a demolding agent, separation of the polyurethane product from the aluminum mold was difficult and the surfaces of the polyurethane product were found to have unacceptable blemishes, gaps, and fissures (Adamovics, unpublished results). After experimenting with several polymeric materials, it was found that the polyurethane formulation of the invention could be efficiently demolded from polypropylene, and that the cured product had high-quality optical surfaces. It was found that a mold fabricated from two polypropylene endplates with one or more polypropylene or polyethylene spacers of known, uniform thickness to function as spacers, placed between the said mold endplates, the whole secured with bolts, provided polyurethane sheets of defined, uniform thickness and high-quality optical surfaces. It will be appreciated that other methods to prepare a dosimetric sheet with dimensions of the sheets of the invention might be used. For example, a rectangular box comprising a bottom face and four side faces of the appropriate x and y dimensions may be fabricated, and a polyurethane formulation of the invention may be admitted to a depth defining the z dimension of the sheet. Careful leveling and curing may provide a suitable dosimetric sheet. This and other known techniques which provide sheets (such as cast rolling or extrusion) are within the scope of the invention.

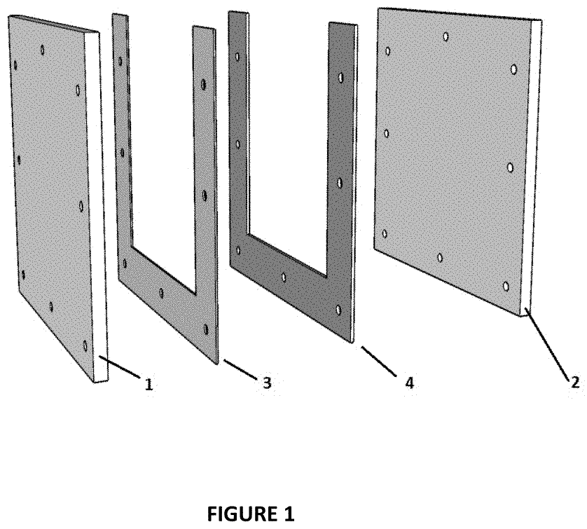

FIG. 1 portrays the components of the dosimeter mold. Polypropylene spacer 3 and optional polypropylene spacer 4, of identical or different thicknesses, are fitted between a first polypropylene endplate 1 and a second polypropylene endplate 2. A variety of polypropylene shims are available, for example from McMaster-Carr (Elmhurst, Ill.). Selection of one or more polypropylene shims fabricated into “U” shaped spacers such as 3 and 4, defines the gap in the assembled mold, and therefore the controlled, uniform thickness of the polyurethane film produced by the mold. The ability of this approach to fabricate sheet dosimeters of desired thicknesses between 0.8 mm to 10.0 mm is determined by the thickness of the spacer or spacers used in the construction of the mold. Polypropylene of various thicknesses to be used as mold endplates or spacers is commercially available. For example, McMaster-Carr (Elmhurst, Ill.), among other vendors, offers a variety of the said polypropylene. As a non-limiting illustration, Table 1 denotes the thicknesses and close tolerances of the said films from McMaster-Carr.

TABLE 1
Polypropylene Shim Films Suitable for Sheet Mold Spacers

| Thickness | | | | |
|---|---|---|---|---|
| Inches | Inches | | Tolerance | |
| Fraction | Decimal | mm | ±in | ±mm |
| 1/32 | 0.031 | 0.794 | 0.004 | 0.102 |
| 3/64 | 0.047 | 1.191 | 0.006 | 0.152 |
| 1/20 | 0.050 | 1.270 | 0.005 | 0.127 |
| 1/16 | 0.063 | 1.588 | 0.007 | 0.178 |
| 5/64 | 0.078 | 1.984 | 0.010 | 0.254 |
| 3/32 | 0.094 | 2.381 | 0.010 | 0.254 |
| ⅛ | 0.125 | 3.175 | 0.013 | 0.330 |

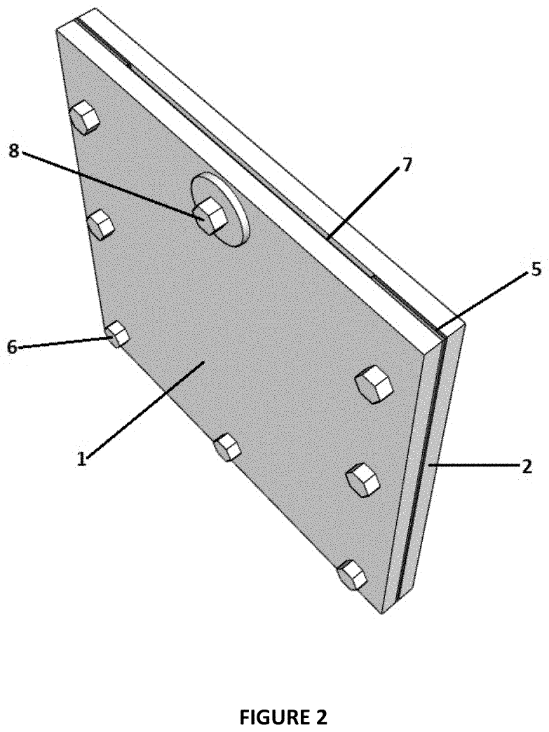

As a non-limiting illustrative example, two polypropylene endplates of ½″ thickness and a polypropylene film (3.18 mm thickness) were precisely machined to have apertures as depicted in FIG. 1. The mold components were assembled to form the mold (FIG. 2). The spacer 5 was placed between a first mold endplate 1 and a second mold endplate 2. The components were secured and compressed together with bolts 6. The assembled mold had a gap 7 of precisely the thickness of the spacer 5 utilized. It will be appreciated that the use of multiple spacers provides a wide range of choices for the sheet formed by the mold. For example, if a sheet of 5 mm thickness is desired, a 1.98 mm spacer and a 3.18 mm spacer could be used in the assembly of the mold. If a 4 mm sheet is needed, two 1.98 mm spacers could be employed. While it is convenient to employ polypropylene as spacers, a variety of other materials which are available in defined and uniform thicknesses may be used. These include, but are not limited to, polyethylene terephthalate glycol-modified, fiberboard, high density polyethylene, Teflon, polycarbonate, and other plastics. The mold endplates 1 and 2 are machined to fit an adjustment bolt 8. After mold assembly, 8 is used to make fine adjustments to the width of gap 7 which may arise from minor deviations of 1 and 2 from planarity. Measurement of gap 7 might be performed with feeler gauges, taper gauges, calipers, or other means to measure spaces in the range of 0.8 mm to 10 mm. One or more polyurethane sheets, previously prepared by the method of the invention and of thickness precisely measure by mechanical or electronic calipers, may also be used for this measurement.

A variant of this mold design affords several sheet dosimeters in a single molding operation. A stacked mold array is assembled in which polypropylene inner plates are added such that the first endplate and the first inner plate are separated by one or more spacers; the first inner plate and the second inner plate are separated by one or more spacers; the second inner plate and the third inner plate are separated by one or more spacers, and so on, and the last inner plate and the second endplate are separated by one or more spacers. When assembled and secured this provides a mold with a plurality of gaps of defined thickness, and after charging with the polyurethane formulation, curing, and demolding, affords a plurality of sheet dosimeters.

Sheet Molding Process. Prepolymer A and Prepolymer B (selected to have a pot-life of 10-20 minutes at 20-25° C.), radical initiator, leuco dye, a first solvent, an optional second solvent, and optional plasticizer are thoroughly mixed and introduced into the sheet mold. The liquid formulation may be admitted to the mold by pouring through a funnel, metering pump, peristaltic pump, or by other means well-known in the art. A preferred method to charge the mold with the liquid formulation by syringe. The mold charged with the formulation is maintained essentially vertically (as in FIG. 2) and placed in a pressure chamber. The chamber is pressurized to between 40 PSI and 120 PSI by any of several means well-known in the art, in order to prevent formation of bubbles in the dosimeter. The chamber might be pressurized by use of compressed gas (for example, air or nitrogen). A preferred method is pressurization with a compressor. The mold is maintained under pressure for the duration of the curing time.

Demolding. When curing is complete, the mold is removed from the pressure chamber and disassembled. The dosimeter product is carefully freed from the mold surface. For light sensitive formulations, a protective film is carefully applied to both faces of the sheet. In some embodiments, the protective film is a dyed polyester color filter which protects the optical surface of the sheets from inadvertent damage in handling. A preferred protective film is GamColor 355 (Rocco, Inc., Stamford, Conn.).

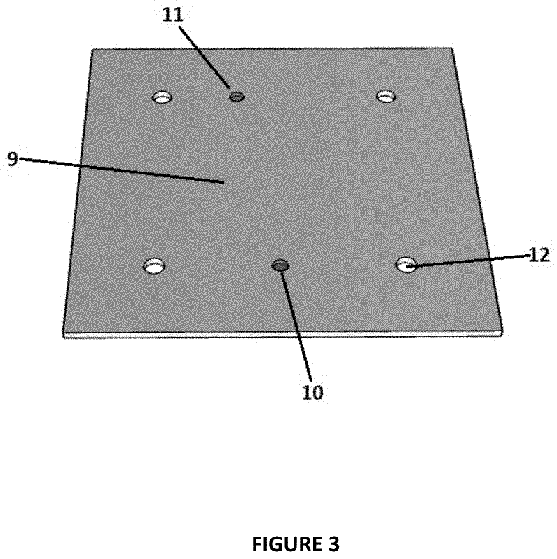

Post-Molding Modification. The dosimeter sheet 9 with a protective film adhered to each surface is tooled to precise length and width dimensions and apertures 12 for assembly bolts are introduced (FIG. 3). In addition, fiducial marks 10 and 11 are engraved. Marks 10 and 11 are designed to enable the precise alignment of individual sheets in the stacking process. After irradiation, fiducial marks on individual irradiated dosimeter sheets ensure proper spatial alignment in the scanning process and proper comparison of scanned images to the treatment plan. The individual sheets may be cut, machined, processed, or modified to have the proper attributes of precise length and width, and precisely placed apertures and fiducial marks by any of several methods well-known in the art. A preferred method of post-molding sheet modification is the use of a laser cutter/engraver. A variety of laser cutters are available and are well-known in the art of laser printing/cutting/engraving. In a non-limiting example, the Glowforge Basic Laser cutter/engraver (Glowforge, Inc., Seattle, Wash.) was used to produce sheet post-molding modifications.

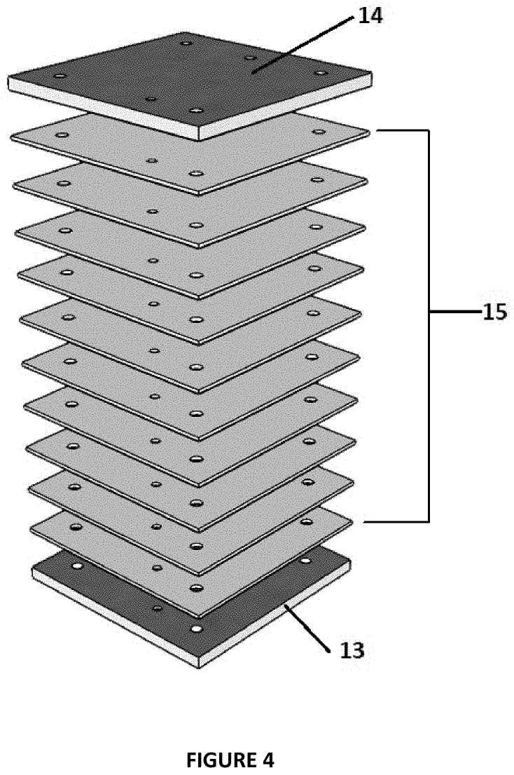

Stack Design and Assembly. For stand alone stacks, the bottom end piece 13 and the top end piece 14 (FIG. 4) of the sheet stack is fabricated from any polymeric tissue-equivalent material available in sheet form. These endpieces are not radiochromic but function to hold the stack of dosimetric sheets together in a precise array. The material for the end pieces can be selected from a number of polymers well-known in the art, including, but not limited to polystyrene, polypropylene, and nylon. A preferred material is polypropylene, which is available from a number of vendors. The end pieces, produced from polypropylene sheets with thickness in the range of ⅛ to ½ are machined to have the same area in the x-y plane as the sheet dosimeters 15. The apertures and fiducial marks are introduced in the same way and in the same orientation as those of the sheets. The end pieces 13 and 14 and the array of dosimetric sheets 15 are arrayed as depicted in FIG. 4. The assembled sheet stack (FIG. 5) is secured and the plurality of dosimetric sheets 15 is compressed between the bottom end piece 13 and the top end piece 14 with nylon bolts 16 and nylon hex nuts (not shown). In some embodiments, the protective film is removed from each individual sheet before assembly into the stack, so that there are no non-dosimetric spacers between the sheets. The fiducial marks 17 and 18 on the top end piece are precisely aligned with those of each individual component sheet. The height of the sheet stack (z-direction, FIG. 5) is determined by the thickness of the individual sheet dosimeters and the number of sheets used to build the stack. Sheet stacks may be made of sheets with thicknesses of between 0.8 mm and 10.0 mm. Sheet stacks may be made by the assembly of between 5 and 100 sheets.

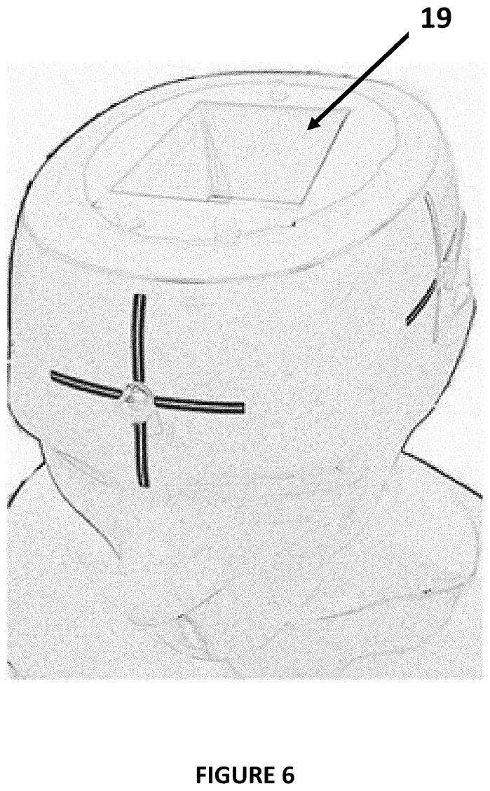

Anthropomorphic phantoms simulate human tissue (L. A. DeWerd and M. Kissick eds., The Phantoms of Medical and Health Physics, New York: Springer, 2014). For dosimetric stacks that are used with phantoms, for example the STEEV head phantom (FIG. 6, CIRS, Norfolk, Va.), the stack of sheet dosimeters with fiducial markings is assembled inserted into the cavity 19. In this and similar applications, the sheets are secured by containment in the phantom cavity and no endpieces or bolts are needed to secure the stack.

Sheet stacks are 3D dosimeters which are capable of measuring essentially the entire target volume of an intended radiation treatment. Table 3 delineates the calculated portion of the height of sheet dosimeter stacks made from ten, twenty, or thirty individual sheets of thicknesses available by molding as described with the polypropylene film spacers described above. Because essentially the entire x-y plane of the stacked sheets is dosimetric material, the total volume of dosimetric media within the sheet stack is identical to the proportion of the height, in the z-direction, of dosimetric material to the total height of the stack composed of sheet dosimeters and protective films. The end pieces of the assembled stack and the protective film between the end pieces and the top and bottom sheets of the stack are not considered as part of the total stack volume.

TABLE 2
Percent total volume of dosimetric media in various Sheet Stacks

| | | | Thick- | Total | | |
|---|---|---|---|---|---|---|
| | | | ness | Height | | |
| | Thick- | Number | of | including | | |
| | ness | of | pro- | pro- | Total | Percent |
| Number | of | pro- | tective | tective | dosimeter | dosimetric |
| of | sheet, | tective | films, | films, | height, | in Total |
| sheets | mm | films | mm | mm | mm | Volume |
| 10 | 1.19 | 18 | 0.05 | 12.8 | 11.9 | 93.0 |
| 20 | 1.19 | 38 | 0.05 | 25.7 | 23.8 | 92.6 |
| 30 | 1.19 | 58 | 0.05 | 38.6 | 35.7 | 92.5 |
| 10 | 1.98 | 18 | 0.05 | 20.7 | 19.8 | 95.7 |
| 20 | 1.98 | 38 | 0.05 | 41.5 | 39.6 | 95.4 |
| 30 | 1.98 | 58 | 0.05 | 62.3 | 59.4 | 95.3 |
| 10 | 3.18 | 18 | 0.05 | 32.7 | 31.8 | 97.2 |
| 20 | 3.18 | 38 | 0.05 | 65.5 | 63.6 | 97.1 |
| 30 | 3.18 | 58 | 0.05 | 98.3 | 95.4 | 97.0 |
| 10 | 3.97 | 18 | 0.05 | 40.6 | 39.7 | 97.8 |
| 20 | 3.97 | 38 | 0.05 | 81.3 | 79.4 | 97.7 |
| 30 | 3.97 | 58 | 0.05 | 122 | 119.1 | 97.6 |
| 10 | 5.16 | 18 | 0.05 | 52.5 | 51.6 | 98.3 |
| 20 | 5.16 | 38 | 0.05 | 105.1 | 103.2 | 98.2 |
| 30 | 5.16 | 58 | 0.05 | 157.7 | 154.8 | 98.2 |

Stacks of sheets with different thicknesses may also be prepared depending on the treatment plan and attributes being evaluated. For example, if greater resolution of the radiation distribution is required a thinner sheet is used while in less critical regions of the treatment plan thicker sheets could be utilized. So, for example, several 5 mm sheets, followed by several 1 mm sheets, followed by several 5 mm sheets might be assembled to capture critical resolution data in the 2 mm sheet region. Stacks with customized sheet thicknesses optimize the time required in verifying a treatment plan.

Post-irradiation. After irradiation with any of a variety of radiation modalities, well-known in the art, the sheet stack is disassembled and each individual sheet is evaluated by 2D scanning. Several flat bed scanners are commercially available, for example the Epson 11000XL. Alternatively, irradiated sheets may be measured by a scanning apparatus consisting of a light source, a lens, and a means to support the sheet. one such apparatus assembled by the inventor comprises a telecentric lens (Promeas DTC23183), c-mount camera (Blackfly, FLIR) with the irradiated dosimetric sheet attached to a red LED array (Metaphase). It is critical that the position of each sheet along the z-axis of the stack is indicated and maintained on each scanned image to ensure proper sequence for a correct rendering of the data. The plurality of 2D scans is compared to the treatment plan by methods well-known in the art, for example by reconstruction into a 3D image by computerized means or by sequential comparison of the 2D images with the radiation treatment plan.

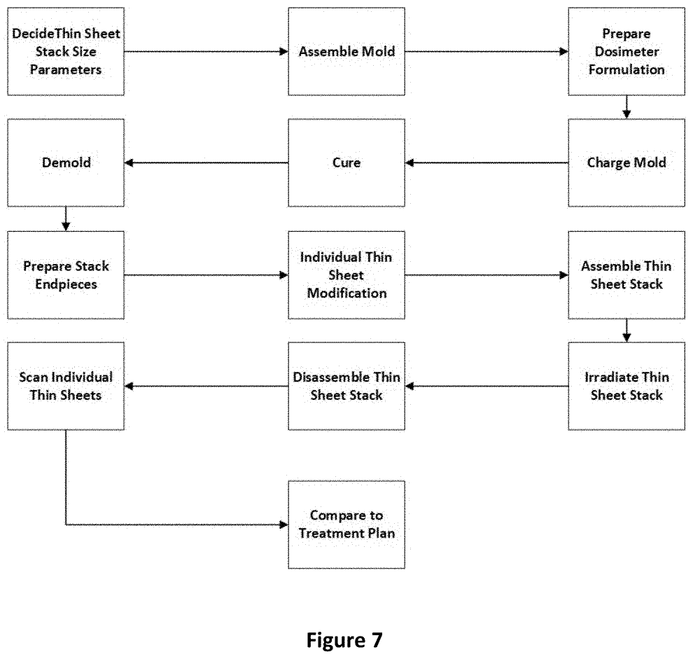

The sequence of steps in the planning, mold construction, formulation, molding, demolding of sheet dosimeters and the assembly and use of sheets in stacks for radiation oncology are summarized in flowchart form in FIG. 7.

### EXAMPLES

Clear polyurethanes were obtained from Crystal Clear 200 from Smooth-On, Easton, Pa., BJB Enterprises Tustin, Calif. or Polytek, Easton Pa. Plastic sheets, silicone cord and shims where obtained from McMaster Carr, Robbinsville, N.J. Solvents and additives were obtained from Sigma Aldrich, St. Louis, Mo. Leuco dyes were synthesized by techniques well known to those with skill in the art.

### Example 1

300 g scale. 2 g LCV was dissolved in 10.5 g dichloromethane (DCM) and 1.5 g carbon tetrabromide. The polyurethane (BJB 780) was softened with 30 g dibutyl phthalate. Samples of sheets were sequentially irradiated to a total dose of 40 Gy using a modified Theratron 60 cobalt radiotherapy machine at dose rates of either 1 or 0.25 Gy per minute. The radiochromic reaction was complete in less than 5 minutes. A linear dose response with a sensitivity of 0.5 cm-1Gy-1 was observed.

### Example 2

260 g polyurethane, 6 g 2-Me-LMG-DEA dissolved in 14 g ethyl acetate, 6 g DMSO and 0.55% carbon tetrabromide were blended in a 0.5 liter container. 10 g dibutyl phthalate was added to soften the sheet. The solution was poured into a 2 mm wide sheet mold. The filled mold was placed under 60 psi pressure for 72 hr. The cured sheet was demolded to provide a 10 cm×10 cm×2 mm sheet with high-quality optical surfaces.

### Example 3

100 g scale. 2 g 4-MeO-LMG-DMA was dissolved in 5 g cyclohexanone and 5 g butyl acetate. 0.55% carbon tetrabromide was dissolved in tetrahydrofuran and 10 g dibutyl phthalate was used as a plasticizer. This solution was blended with 79 g polyurethane. The solution was poured into a 2 mm wide sheet mold. The filled mold was placed under 60 psi pressure for 24 hr.

### Example 4

A 2 mm sheet was formulated with 4 g 2-Me-LMG-DEA dissolved in 8 g DCM and 8 g toluene which was blended with 20 g dibutyl phthalate, 1.5 g carbon tetrabromide and 178 g polyurethane. The sheet was irradiated with a treatment plan delivering 8 Gy and matched to the expected result to 96.4%

### Example 5

The effects of temperature on the proton Bragg peak was investigated using a 5 mm sheet. The 350 g formulation was composed of 42 g cumin-LMG-DEA dissolved in 96 g cyclohexanone with 420 g of a softener (CAS 474919-59-0). This was blended with 31.2 g carbon tetrabromide and 1535 g BJB polyurethane.

### Example 6

After the sheets are irradiated, they are scanned on an Epson 11000XL high-resolution scanner and the acquired optical density (OD) information is analyzed by Image J (https://imagej.nih.gov/). The measured distributions were compared to the calculated distributions from the treatment planning system, (Eclipse, Varian Inc.) at each depth, simulated images of the dosimeter were imported into the planning system to perform dose calculation. The passing rates of gamma tests between dose distributions from EBT3 film (Ashland Specialty Ingredients, Bridgewater, N.J., USA) and PRESAGE sheets, EBT3 and the treatment planning system at eight different depths were calculated using Dose Lab (Mobius Inc.). The stacks are 3D volume rendered using ImageVis3D (http://www.sci.utah.edu/software/imagevis3d.html) and 3D reconstruction using 3D slicer (https://www.slicer.org/).

### Example 7

A pliable sheet formulation: 1% LCV, 7% DCM, 20% dimethyl phthalate, 1% CBr4, 1% cyclohexanone curing time of 24 hr at 25° C.

### Example 8

Sheets for the anthropomorphic phantom STEEV. Sheet formulation: 2% 2,4 dimethyl-LMG-DMA, 4% 1-methyl-2-pyrrolidinone, 2% 1,2,4-trimethylbenzene, 0.5% carbon tetrabromide, BJB polyurethane poured into 30 cm×30 cm by 3 mm wide molds and cured for approximately 24 hr. The demolded 3 mm thick sheets were laser cut into 6 cm×6 cm squares with each containing two engraved fiducial markers. Nineteen sheets were inserted into the STEEV phantom (FIG. 6) which was irradiated with a Gamma Knife (Elekta AB, Stockholm, Sweden) treatment plan for an acoustic neuroma. After the sheets were irradiated, they were scanned on an Epson 11000XL high-resolution scanner and the acquired OD information was analyzed by an application in the public domain, Image J (https://imagej.nih.gov/) and compared to the treatment plan. An alternative open-source software used to process 2D scans is 3D slicer (https://www.slicer.org/). Additionally, a commercially available software program may be used (https://analyzedirect.com/).

### Example 9

Sheet uniformity. Sheets of approximately 3 mm thick, 1 cm wide×35 cm long were fabricated with a formulation of 1% LCV, 7% DCM, 10% dimethyl phthalate, 1% CBr4, 1% cyclohexanone curing time of 24 hr at 25° C. After irradiation the dose uniformity had a relative standard deviation of 1.4%.

1. A sheet dosimeter having a first face and a second face essentially parallel to said first face,
wherein each face has the same length and width, and a thickness; and
wherein said length is between 10 and 25 cm and said width is between 1 and 25 cm, and said thickness is between 0.8 and 10.0 mm; and
wherein said dosimeter is prepared from a formulation of a polyurethane prepolymer A, a polyurethane prepolymer B, a leuco dye, and additives selected from the group consisting of radical initiator, first solvent, second solvent, plasticizer, and softener; and
wherein said sheet dosimeter is caused to have fiducial markings.

2. A sheet dosimeter of claim 1 wherein said formulation is introduced into a mold comprising a first endplate, one or more spacers, and a second endplate, and said spacers are made to fit between the said endplates, and said endplates and spacers are caused to be joined together, said spacers causing a gap between said endplates, said gap being between 0.8 to 10.0 mm, said gap determining the thickness of the dosimeter; and
wherein said formulation is allowed to cure within the mold held in essentially a vertical position at 20-30 degrees C. under 40-120 PSI pressure for between 8 hours and 7 days; and
wherein the cured dosimeter is freed from said mold by the disassembly of said mold; and
wherein said first face and said second face are high-quality optical surfaces.

3. A sheet dosimeter assembly comprised of a plurality of sheets of claim 1 caused to be arrayed and in a stack, wherein said stack is comprised of between 5 and 100 sheets.

4. A method in which the dosimeter of claim 1 is used for in vivo dosimetry.

5. A method of evaluating a radiation treatment plan wherein the stack of claim 3 is subjected to a field of radiation; and
wherein said irradiated stack is disassembled to provide a plurality of individual irradiated sheet dosimeters; and
wherein the optical changes in each individual sheet dosimeter are measured and compared to a radiation treatment plan.
